(19) United States
(12) Patent Application Publication
(10) Pub. No.: US 2019/0090776 A1
(43) Pub. Date: Mar. 28, 2019

(54) IMPROVED NEUROLOGICAL FEEDBACK DEVICE

(71) Applicant: Inria Institut National De Recherche En Informa..., Le Chesnay (FR)

(72) Inventors: Marsel Mano, Rennes (FR); Christian Barillot, Laillé (FR); Anatole Lecuyer, Rennes (FR)

(21) Appl. No.: 16/085,166

(22) PCT Filed: Mar. 14, 2017
(86) PCT No.: PCT/FR2017/050567

§ 371 Date: Sep. 14, 2018

(30) Foreign Application Priority Data
Mar. 17, 2016 (FR) 1652279

(51) Int. Cl. A61B 5/0482; A61B 5/00; A61B 5/0205; A61B 5/026; A61B 5/04; G16H 30/20; G16H 10/60; G16H 20/40

(52) U.S. Cl. CPC A61B 5/0482; A61B 5/0042; A61B 5/0205; A61B 5/0263; A61B 5/055; A61B 5/04012; G16H 30/20; G16H 10/60; G16H 20/40; A61B 5/742

(57) ABSTRACT

The invention relates to a neurological feedback device comprising a memory (4) for receiving electroencephalogram data and raw functional and metabolic magnetic resonance imaging data, an electroencephalogram block (10) comprising at least one electroencephalogram processing unit (14, 16, 18) arranged so as to process raw electroencephalogram data and to calculate a value linked to a brain wave, a functional magnetic resonance imaging block (20) comprising at least one functional and metabolic magnetic resonance imaging processing unit (24, 26, 28) arranged so as to process raw functional magnetic resonance imaging data and to calculate a value of perfusion or oxygenation of certain zones of the brain, a synchroniser (30) arranged so as to synchronise temporally the electroencephalogram data and raw functional magnetic resonance imaging data and the processing thereof in the electroencephalogram block (10) and the functional magnetic resonance imaging block (20), and a calculator (40) arranged so as to determine a score representing a correlation between values associated with a neurological rehabilitation activity and values generated by the electroencephalogram block (10) and the functional magnetic resonance imaging block (20), which are obtained from electroencephalogram and functional magnetic resonance imaging measurements of a patient seeking to reproduce the neurological rehabilitation activity, the device being arranged so as to emit neurological feedback to the patient on the basis of said score.

The invention relates to the field of clinical neuroscience and in particular to neurological feedback devices.

When patients have lost brain control abilities, for example following a stroke, neurorehabilitation is a long and important step on their path to healing.

This rehabilitation is especially complex to implement given that the brain in its entirety remains poorly understood, in particular as regards learning mechanisms.

Present day techniques in the field of neurological feedback are mainly based on electroencephalogram (EEG below) or functional and metabolic magnetic resonance imaging (fMRI below) measurements. This technique consists mainly in the alternation of periods of the chosen rehabilitation activity, during which an EEG or fMRI measurement is carried out, with the display of feedback based on the measurement, and periods of relaxation before the repetition of the chosen rehabilitation activity.

The invention aims to improve the situation. To this end, the applicant proposes a neurological feedback device comprising a memory able to receive raw functional and metabolic magnetic resonance imaging data and electroencephalogram data, an electroencephalogram block comprising at least one electroencephalogram processing unit arranged to process raw electroencephalogram data and to compute a value related to a brainwave, a functional magnetic resonance imaging block comprising at least one functional and metabolic magnetic resonance imaging processing unit arranged to process raw functional magnetic resonance imaging data and to compute an oxygenation or perfusion value of certain brain zones, a synchronizer arranged to temporally synchronize the raw functional magnetic resonance imaging data and electroencephalogram data and their processing in the electroencephalogram block and the functional magnetic resonance imaging block, and a computer arranged to determine a score representing a correspondence between values associated with a neurorehabilitation activity and values from the electroencephalogram block and the functional magnetic resonance imaging block and obtained from functional magnetic resonance imaging and electroencephalogram measurements on a patient seeking to reproduce the neurorehabilitation activity, the device being arranged to provide neurological feedback to the patient on the basis of said score.

This type of device is particularly advantageous because it allows feedback based on two types of measurement of different and potentially complementary nature to be combined together, this allowing a more effective rehabilitation to be carried out.

According to variant embodiments, the device will possibly have one or more of the following features:
the computer is arranged to control a display depending on the computed score,
the computer is arranged to control the display during a chosen duration corresponding to a period during which a patient is seeking to reproduce the neurorehabilitation activity,
the computer is arranged to compute said score during a period of 5 seconds to 1 minute, preferably 20 seconds, during which a patient is seeking to reproduce the neurorehabilitation activity,
the computer is arranged to update data for computing the score during a chosen duration corresponding to a period of rest for the patient,
the computer is arranged to update data for computing the score during a chosen duration of 5 seconds to 1 minute, preferably 20 seconds, corresponding to a period of rest for the patient,
the device furthermore comprises an electroencephalogram sensor,
the device furthermore comprises a functional magnetic resonance imaging sensor.

Other features and advantages of the invention will become more clearly apparent on reading the following description given by way of nonlimiting illustrative example with reference to the drawings, in which:

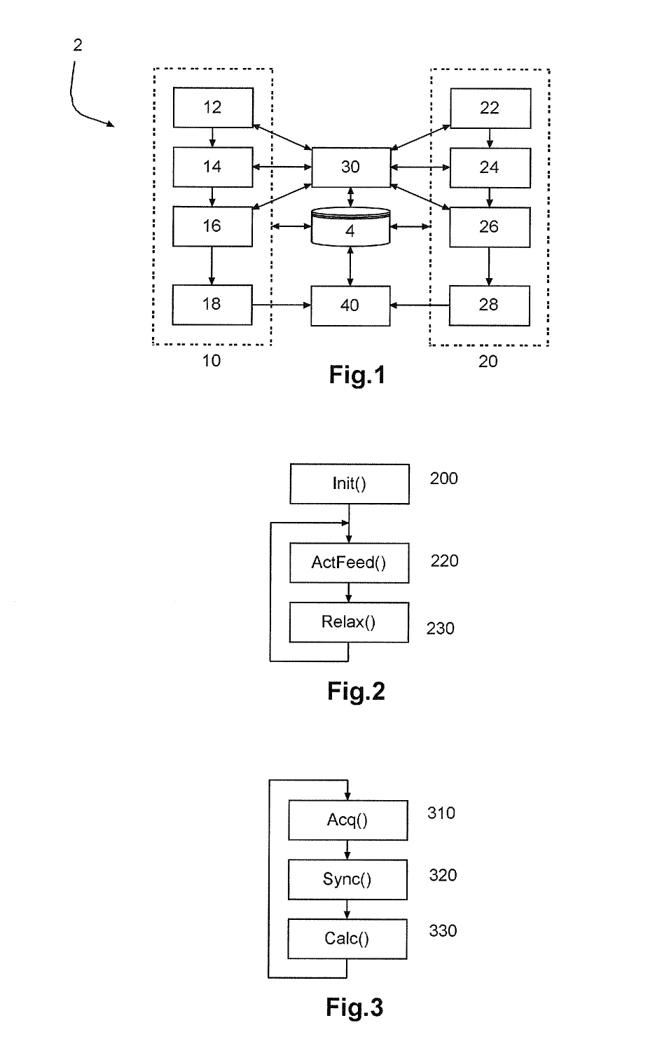

FIG. 1 shows a schematic diagram of a device according to the invention,

FIG. 2 shows an example of implementation of the device of FIG. 1,

FIG. 3 shows an example of implementation of a function by the block 10 or the block 20 of FIG. 1,

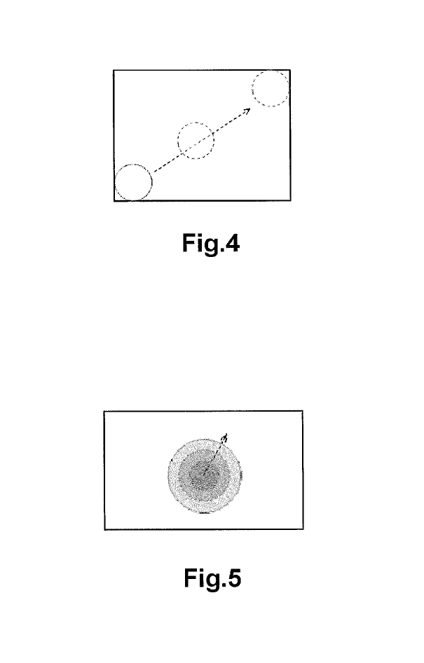

FIG. 4 shows an example of neurological feedback provided by the device according to the invention,

FIG. 5 shows another example of neurological feedback provided by the device according to the invention.

The drawings and description below contain, for the most part, elements of a definite nature. They will therefore not only serve to better understand the present invention, but may also, where appropriate, contribute to its definition.

The present description is of a nature to involve elements subject to protection by authors' rights and/or copyright. The owner of these rights has no objection to anyone producing an identical copy of the present patent document, or of its description as it appears in the official records. In all other respects, he reserves his rights in their entirety.

FIG. 1 shows a schematic diagram of a device 2 according to the invention. The device 2 comprises a memory 4, an EEG block 10, an fMRI block 20, a synchronizer 30, and a computer 40.

In the context of the invention, the memory 4 may be any type of data storage able to receive digital data: hard disk, solid state drive (SSD), flash memory in any form, random access memory, magnetic disk, distributed storage located locally or in the cloud, etc. The data computed by the device may be stored on any type of memory similar to the memory 2, or on said memory. These data may be erased after the device has carried out its tasks or preserved.

In the example described here, the EEG block comprises an EEG sensor 12 and EEG processing units 14, 16 and 18. The processing units 14 and 16 are dedicated to denoising/resampling, and to power estimation, respectively, whereas the processing unit 18 uses a data model to determine characteristics, based on the power estimation, such as alpha and beta waves. The units 14 to 18 are connected in series, such that an image from the EEG sensor 12 is processed in succession by said units so as to obtain a result. As a variant, some of these units could be grouped together, others omitted, or indeed other units could be added, and the sensor 12 could be considered not to form part of the device 2.

In the example described here, the fMRI block 20 comprises an fMRI sensor 22 and fMRI processing units 24, 26 and 28. The processing unit 24 is dedicated to realigning the image in order to take into account movements of the patient during the image capture. The processing unit is dedicated to processing the realigned image in order to correct the biases due to the slicing of the fMRI process. Lastly, the processing unit 28 uses a data model to determine activation characteristics of zones of the brain, based on the data from the processing unit 26. The units 24 to 28 are connected in series, such that a series of 3-D images from the fMRI sensor 22 is successively processed by these units to obtain a result. As a variant, some of these units could be grouped together, other units could be added, and the sensor 22 could be considered not to form part of the device 2.

The EEG processing units 14 to 18, the fMRI processing units 24 to 28, the synchronizer 30 and the computer 40 are elements able to access the memory 4 directly or indirectly. They may take the form of a suitable computer code executed by one or more processors. The term “processor” must be understood to mean any processor suitable for processing imaging data and for synchronizing temporally marked data. Such a processor may take any known form, such as a personal computer microprocessor, a dedicated FPGA or SOC (system on chip), a networked computational resource, a microcontroller, or any other form able to deliver the computational power required by the embodiment described below. One or more of these elements may also take the form of specialized electronic circuits such as an ASIC. A combination of a processor and of electronic circuits may also be envisioned.

The two blocks 10 and 20 not only relate to measurements that are of completely different type, but they furthermore have very different processing and acquisition frequencies. Indeed, the sensor 12 typically has an acquisition frequency of about 5 kHz, and its output is generally resampled at 250 Hz, whereas the sensor 22 emits about one 3-D image per second on average.

The fundamental difference in rate and the a priori different nature of the data obtained from these measurements has up to now dissuaded the man skilled in the art from seeking to combine EEG and fMRI together to carry out neurological rehabilitation.

The applicant has discovered that the data obtained from EEG and fMRI, and their synchronization, allows remarkable results to be obtained. The function of the synchronizer 30 is thus to temporally align the data in the EEG block 10 on the one hand and in the fMRI block 20 on the other hand, and the computer 40 allows a score combining the synchronized measurements obtained from these blocks to be computed.

FIG. 2 shows an example of how the device according to the invention may function. In a first operation 200, the device is initialized by a function Init( ). This function initializes the blocks 10 and 20, and the synchronizer 30 and the computer 40, and furthermore controls, in the example described here, a display for the interaction with the patient. Thus, during the initialization period, a representation of the task to be carried out must be presented and explained to the patient, and the latter will be invited to carry out this task a first time.

Next, a loop is initialized in which the patient is invited to reproduce the task again in an operation 210, then to rest during an operation 220.

During the operation 210, the device executes a function ActFeed( ) in which the blocks 10 and 20 measure the brain activity of the patient and the computer 40 computes a score reflecting feedback of the neurological activity of the patient during the execution of the required task. This feedback is displayed simultaneously, so that the patient may concentrate on the way of stimulating his brain that optimizes the displayed feedback.

The score from the EEG block 10 may be based on an evaluation of the power of the EEG signals in different frequency bands (also called band power in the art) during the execution of the task that the patient is asked to carry out. Indeed, a given task may be known to cause brain stimulation taking the form of brainwaves in one or more frequency bands, and the EEG block 10 determines the correspondence between the frequencies of excitation of the brain of the patient and the targeted band power. Typically, the sought-after waves will possibly be delta waves (between 0.5 and 4 Hz) theta waves (between 4 and 8 Hz), alpha waves (between 8 and 13 Hz), and beta waves (between 13 and 30 Hz), or even peaks at 3 Hz.

The score from the fMRI block 20 may be based on an evaluation of the amount of oxygen in a region of interest of the brain. Indeed, a given task may be known to induce brain activity that results in oxygenation of a specific region, and the fMRI block 20 determines the correspondence between the excited region of the brain of the patient and the target zone.

The computer 40 is arranged to combine the scores from the block 10 and the block 20, respectively, and to present them in a relevant way to the patient.

According to a first variant shown in FIG. 4, the computer 40 may use the respective scores as coordinates in a two-dimensional plane, and display visual feedback in which the bottom left-hand corner of the image represents negative feedback and the top right-hand corner of the image represents positive feedback, the progression along either one of the axes indicating that the EEG or fMRI score is improving. By positive feedback what is meant is the fact that the scores indicate a brain activity corresponding to the expected activity, and by negative feedback what is meant is brain activity not corresponding to the expected activity.

According to a second variant shown in FIG. 5, the computer 40 may use the respective scores as a size and a color for a represented shape, and display visual feedback in which the size of the shape increases as the EEG score (or fMRI score, respectively) improves and in which the color of this shape may vary from blue to red depending on the fMRI score (or EEG score, respectively), a blue color indicating a low score and a red color indicating a high score.

Many other variants are envisionable, in particular a logarithmic, or binary, or stepwise variation of the representation of the scores.

As a variant, it is the computer 40 and not the block 10 and the block 20 that determines the correspondence between the measurements and values associated with the rehabilitation activity.

During the operation 220, the device is arranged to continue to analyze the data from the blocks 10 and 20, in order to adjust the following repetition of the operation 210. This operation may in particular include the definition of new comparison thresholds for the computation of the respective scores and/or their processing by the computer 40.

FIG. 3 shows an example of how the block 10 (the block 20, respectively) may function. The block 10 executes a loop starting with an operation 310 in which the EEG measurement (or fMRI measurement, respectively) is acquired by the sensor 12 (from the sensor 22, respectively).

Next, in an operation 320, the block 10 (the block 20, respectively) interacts with the synchronizer 30 in order to temporally realign the data from the sensor 12 (from the sensor 22, respectively).

The synchronization of the data is essential. Indeed, the neurological feedback processing operations measure extremely modest signal increases, about 1% for the brain regions targeted, for example in the case of patients having suffered a stroke. It is crucial for the measurement signals to be correctly synchronized, in order not to compute scores based on data that are temporally unrelated, which would be less relevant than separate scores.

This synchronization is carried out using temporal markers associated with each signal, these temporal markers moreover having a common temporal reference. In order not to disrupt the signal acquisition devices, raw signals are recorded, and a function allows to analyze the acquired signals by searching for particular temporal markers.

During the real-time computation, another synchronization layer is achieved using the common temporal reference to select only relevant data. For example, in the case of neurological feedback during which a given task is carried out a plurality of times, interspersed by breaks, the time intervals associated with each period of activity or of rest are known beforehand, and the data are sliced directly in the buffers on the basis of the common temporal reference. In contrast, in the case of neurological feedback in which a patient is asked to perform an activity until given neurological feedback is achieved in a given time window, the synchronization is carried out at regular intervals, preferably at a multiple of the highest acquisition frequency.

Lastly, in an operation 330, the block 10 (the block 30, respectively) processes the temporally realigned data in the processing units 14 to 18 (the processing units 24 to 28, respectively).

As may be seen in FIG. 1, in the example described here, the synchronizer 30 also interacts with the processing units 14 and 16 (the processing units 24 and 26, respectively). As a variant, this interaction will possibly be omitted. Conversely, as a variant, the processing unit 18 (the processing unit 28, respectively) will possibly also interact with the synchronizer 30.

1. A neurological feedback device comprising a memory able to receive raw functional and metabolic magnetic resonance imaging data and electroencephalogram data, an electroencephalogram block comprising at least one electroencephalogram processing unit arranged to process raw electroencephalogram data and to compute a value related to a brainwave, a functional magnetic resonance imaging block comprising at least one functional and metabolic magnetic resonance imaging processing unit arranged to process raw functional magnetic resonance imaging data and to compute an oxygenation or perfusion value of certain brain zones, a synchronizer arranged to temporally synchronize the raw functional magnetic resonance imaging data and electroencephalogram data and their processing in the electroencephalogram block and the functional magnetic resonance imaging block, and a computer arranged to determine a score representing a correspondence between values associated with a neurorehabilitation activity and values from the electroencephalogram block and the functional magnetic resonance imaging block and obtained from functional magnetic resonance imaging and electroencephalogram measurements on a patient seeking to reproduce the neurorehabilitation activity, the device being arranged to provide neurological feedback to the patient on the basis of said score.

2. The device as claimed in claim 1, wherein the computer is arranged to control a display depending on the computed score.

3. The device as claimed in claim 2, wherein the computer is arranged to control the display during a chosen duration corresponding to a period during which a patient is seeking to reproduce the neurorehabilitation activity.

4. The device as claimed in claim 3, wherein the computer is arranged to compute said score during a period of 5 seconds to 1 minute, during which a patient is seeking to reproduce the neurorehabilitation activity.

5. The device as claimed in claim 1, wherein the computer is arranged to update data for computing the score during a chosen duration corresponding to a period of rest for the patient.

6. The device as claimed in claim 5, wherein the computer is arranged to update data for computing the score during a chosen duration of 5 seconds to 1 minute, corresponding to a period of rest for the patient.

7. The device as claimed in claim 1, furthermore comprising an electroencephalogram sensor.

8. The device as claimed in claim 1, furthermore comprising a functional magnetic resonance imaging sensor.

9. The device as claimed in claim 4, wherein the computer is arranged to compute said score during a period of 20 seconds, during which a patient is seeking to reproduce the neurorehabilitation activity.

10. The device as claimed in claim 6, wherein the computer is arranged to update data for computing the score during a chosen duration of 20 seconds.
